(19) United States
(12) Patent Application Publication
(10) Pub. No.: US 2011/0137141 A1
(43) Pub. Date: Jun. 9, 2011

(54) Wireless Monitoring of Multiple Vital Signs

(75) Inventors: Leonid V. Razoumov, Bronx, NY (US); Robert R. Miller, II, Convent Station, NJ (US)

(73) Assignee: AT&T INTELLECTUAL PROPERTY I, L.P., Reno, NV (US)

(21) Appl. No.: 12/630,274

(22) Filed: Dec. 3, 2009

(51) Int. Cl. A61B 5/1455

(52) U.S. Cl. 600/316; 600/324

(57) ABSTRACT

Devices, systems and methods are presented for monitoring a plurality of vital statistics, sending the statistics over a communications network, and generating real-time feedback for the user. A device includes a microcontroller, a transceiver, a plurality of logic units, a plurality of transducers, and a plurality of sensors. The sensors measure a plurality of vital statistics for a user. Alerts are communicated to the user via a speaker. Audio signals are detected via a microphone and processed by the logic. The transceiver enables wireless communication, directly or across a network. The transceiver further communicates with a wireless communication device. The plurality of health sensors includes a thermometer, a pulse oximeter, and a blood glucose meter.

### BACKGROUND OF THE INVENTION

1. Field of the Invention

The present invention relates to health monitoring. More particularly, the present invention relates to wireless in-ear health monitoring devices and methods.

2. Background of the Invention

Visiting a doctor is often important to ensure the health of an individual, not only when the individual is sick, but also for regular check-ups. However, doctor visits can be costly, time consuming, and sometimes unpleasant. Many illnesses require constant visits to the doctor for monitoring. This monitoring may simply be monitoring an individual's blood pressure, determining if any changes have occurred, etc. Unfortunately, to receive this monitoring, the individual may have to drive a long distance, sit in a waiting room, see the doctor, and then drive the long distance home. This is not ideal, as it may end up taking most of, if not all of, the day. Also, certain medical conditions require rapid response to changes in vital signs. Real-time continuous and automated monitoring, with proper real-time notification and alerting capabilities, could improve quality of healthcare and reduce its cost.

Today there are many health statistics that are important to diagnosing the average individual. Besides statistics like pulse and temperature, blood oxygen levels provide diagnoses into abnormal ventilation and other respiratory and cardiac problems. Further, as the diabetes epidemic rises, blood sugar is an increasingly important metric for many individuals. Frequent measurements of blood glucose are essential for diabetic patients without adequate glycemic control.

When visiting the doctor, people often complain about past conditions or episodes. However, a doctor can only test the patient's current status and ask them questions to recall how they felt during the past episode. Patient accounts can be uninformative and unreliable. Patients largely do not recall things such as instant pulse, blood pressure, temperature, etc. For instance, a patient may remember feeling cold, which can indicate a high temperature, but there is no way for the doctor to determine the exact temperature or even if the patient had a fever at all.

Many individuals would much rather stay in the privacy of their own home. However, the cost of frequent house calls by a doctor or other health care professional is too much for most individuals. Thus, staying at home is currently not a real option. Devices have been created that allow for remote monitoring of certain vital statistics and transmittal of these vital statistics to a remote server at a medical station for diagnosis and feedback. However, existing devices and methods do not adequately address the needs of the average individual who requires regular feedback, particularly in the case of diabetics.

What is needed is a streamlined portable non-intrusive device that enables measurement and communication of vital statistics across a network and provides real-time feedback to the user of the device.

### SUMMARY OF THE INVENTION

The present invention addresses the above problems by providing devices, systems, and methods for conveniently monitoring a plurality of vital statistics, and generating real-time feedback for the user. This is achieved by incorporating a plurality of sensors into a headset adapted to be worn by a user over the user's ear. The headset also includes a processor, memory, and transceiver. The headset can be used to communicate via a network, or via a wireless communication device such as a cellular telephone.

In one exemplary embodiment, the present invention is a wireless in-ear health monitoring device, including a processor, a memory, and a transceiver configured to communicate over a wireless network. The device includes a plurality of health sensors coupled to the device, the plurality of health sensors measuring a corresponding plurality of vital statistics, a sensing logic on the memory to operate the plurality of health sensors and to receive/send the corresponding plurality of vital statistics from the plurality of health sensors, a communication logic on the memory to operate a microphone and a speaker coupled to the device, and a hearing aid logic on the memory to process an incoming audio signal. The plurality of health sensors includes a thermometer, a pulse oximeter, and a blood glucose meter.

In another exemplary embodiment, the present invention is a system for monitoring health of a user. A microcontroller includes a processor, a memory, a battery, and a transceiver. A plurality of health sensors are in communication with the microcontroller, the plurality of health sensors measuring a corresponding plurality of vital statistics for the user. The system includes a headset in communication with the microcontroller, the headset including a speaker and a microphone, a hearing aid logic to process an incoming audio signal based upon a hearing profile for the user, and an alerting logic in communication with the microcontroller to generate an alert via the speaker when one of the plurality of vital statistics exceeds a maximum or minimum threshold. The transceiver is configured to allow communication with a wireless telecommunication device. The plurality of health sensors includes a thermometer, a pulse oximeter, and a blood glucose meter. The sensor measurements can be processed in the microcontroller or at the wireless telecommunication device and/or on the remote server.

In yet another exemplary embodiment, the present invention is a method for health monitoring, including providing a headset to be worn by a user, the headset including a speaker and a microphone, enabling communication between the headset and a wireless communication device operated by the user, incorporating a plurality of health sensors into the headset, the plurality of health sensors measuring a corresponding plurality of vital statistics for the user, alerting the user via the speaker if any of the plurality of vital statistics exceeds a maximum or minimum threshold, processing an incoming audio signal based on a listening profile for the user, and outputting the processed audio signal via the speaker. The method further includes transmitting the plurality of vital statistics to a remote server for further processing.

### BRIEF DESCRIPTION OF THE DRAWINGS

FIG. 1 shows the components of a health monitoring device, according to an exemplary embodiment of the present invention.

FIG. 2 shows a health monitoring device, according to an exemplary embodiment of the present invention.

FIG. 3 shows a system for monitoring health, according to an exemplary embodiment of the present invention.

FIG. 4 shows a method for monitoring health, according to an exemplary embodiment of the present invention

### DETAILED DESCRIPTION OF THE INVENTION

The following detailed description presents systems and methods for monitoring a plurality of vital statistics, and generating real-time feedback for the user. A device includes a microcontroller, a transceiver, a plurality of logic units, a plurality of transducers, and a plurality of sensors. The sensors measure a plurality of vital statistics for a user. Alerts are communicated to the user via a speaker. Audio signals are detected via a microphone and processed by the logic. The transceiver enables wireless communication, directly or across a network. The transceiver further communicates with a wireless communication device.

“Wireless communications device”, as used herein and throughout this disclosure, refers to any device capable of sending and receiving data across a network. Examples of wireless communications devices include cellular telephones, personal digital assistants (PDAs), laptop computers, portable music devices having transceivers, residential gateways, relays, mesh network forwarding nodes, etc.

A microcontroller includes, inter alia, a memory and a processor. The memory stores applications, software, or “logic units” as a set of instructions or a computer program. “Logic”, as used herein and throughout this disclosure, refers to any information having the form of instruction signals and/or data that may be applied to direct the operation of a processor. Examples of processors are computer processors (processing units), microprocessors, digital signal processors, controllers and microcontrollers, etc. Logic may be formed from signals stored in a device memory. Software is one example of such logic. Examples of device memories that may comprise logic include RAM (random access memory), flash memories, ROMS (read-only memories), EPROMS (erasable programmable read-only memories), and EEPROMS (electrically erasable programmable read-only memories). Logic may also be comprised by digital and/or analog hardware circuits, for example, hardware circuits comprising logical AND, OR, XOR, NAND, NOR, and other logical operations. Logic may be formed from combinations of software and hardware. On a network, logic may be programmed on a server, or a complex of servers. A particular logic unit is not limited to a single logical location on the network.

Communication devices communicate with each other and with other elements via a communication network, for instance, a wireless network, or a wireline network. Networks can include broadband wide-area networks or local-area networks. Communication across a network is preferably packet-based; however, radio and frequency/amplitude modulations networks can enable communication between communication devices using appropriate analog-digital-analog converters and other elements. Examples of radio networks include Wi-Fi, ZIGBEE™, and BLUETOOTH® networks, with communication being enabled by hardware elements called “transceivers.” A network typically includes a plurality of elements that host logic for performing tasks on the network. In modern packet-based wide-area networks, servers may be placed at several logical points on the network. Servers may further be in communication with databases and can enable communication devices to access the contents of a database.

A transducer is any device capable of converting acoustic energy into electrical energy, and vice versa. Transducers include sound generators such as speakers, and sound detectors such as microphones. Most transducers are able to handle audio frequencies of between 10 Hz and 20 kHz; however, different transducers have different acoustic profiles. Digital signal processing (DSP) logic can further be used to tune the output of a transducer.

A sensor is a device capable of detecting the presence or level of an environmental condition, and generating a corresponding signal. An example of a sensor is a medical sensor. The environment would be the human body, or a particular organ, and the environmental condition would be a vital statistic. Medical sensors therefore generate signals corresponding to vital statistics. Sensors can include processing logic onboard the sensor, as well as an interface enabling communication with other processors.

Embodiments of the present invention are illustrated with reference to the figures. For the following description, it can be assumed that most correspondingly labeled structures across the figures (e.g., 132 and 232, etc.) possess the same characteristics and are subject to the same structure and function. If there is a difference between correspondingly labeled elements that is not pointed out, and this difference results in a non-corresponding structure or function of an element for a particular embodiment, then that conflicting description given for that particular embodiment shall govern.

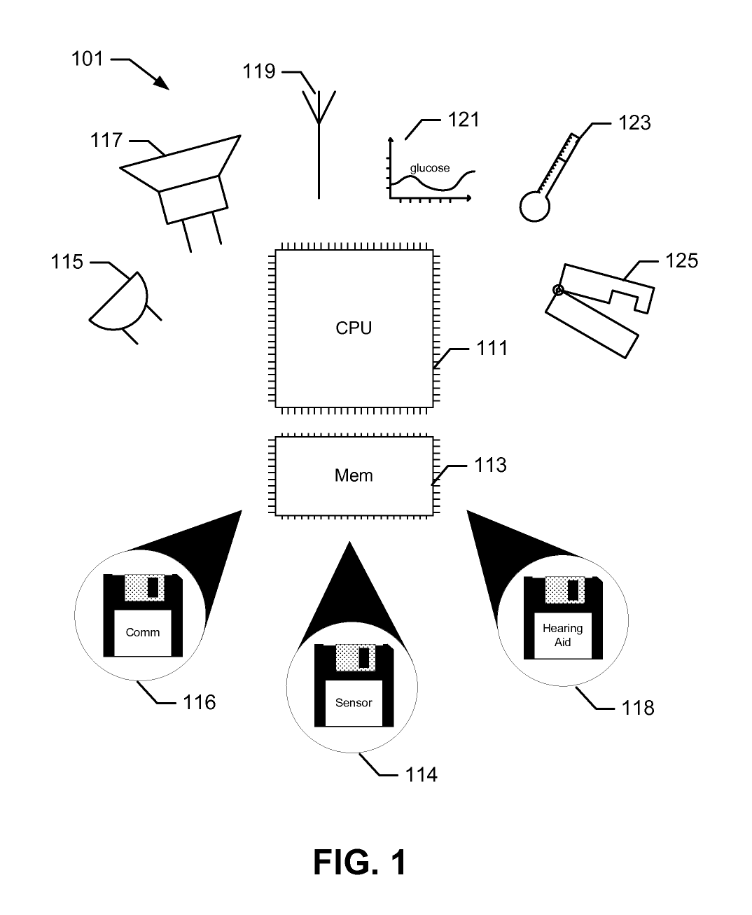

FIG. 1 shows the components of a device 101 for monitoring a user's health, according to an exemplary embodiment of the present invention. Device 101 includes a processor (CPU) 111 and a memory 113. Device 101 also includes a microphone 115, a speaker 117, a transceiver 119, and a plurality of sensors 121, 123, and 125. Memory 113 includes sensor logic 114, communication logic 116, and hearing aid logic 118. Processor 111 and memory 113, together with logic units 114, 116, and 118 may be part of a microcontroller. These components are designed to be compact, and discreetly enclosed in a device designed to be worn over a user's ear. Consequently, at least microphone 115 and speaker 117 are housed in, for instance, a wearable earpiece or headset. Processor 111, memory 113, and transceiver 119 are also enclosed in the headset housing, along with a discreet antenna. Furthermore, at least one of sensors 121, 123, or 125 may be enclosed within the headset housing. For instance, the headset has an earpiece, where speaker 117 is a small diaphragm which, along with an in-ear thermometer 123 is adapted to be inserted into a user's ear canal. Other configurations of sensors 121, 123, and 125 will become apparent in light of the following disclosure.

Sensors 121, 123, and 125 measure vital statistics from a user's body, particularly an ear canal or ear lobe, and generate an electrical signal. For instance, in-ear thermometer 123 detects a user's temperature and generates a corresponding signal. Sensor 121 periodically measures a blood glucose level in any compact non-invasive manner known in the art, such as spectroscopy, and generates a corresponding signal. Sensor 125 is a pulse oximeter that includes an optical sensor to measure blood oxygen and pulse, and may clip onto an ear lobe. Sensor logic 114 enables the microcontroller to receive and process signals from each of the sensors. Each sensor may further have its own logic. Each signal corresponds to a particular vital statistic, in this instance, blood sugar, temperature, pulse, and blood oxygen. Sensor logic 114 can also include alerting logic configured to alert the user as to the status of each signal. For instance, alert logic can be programmed to alert the user if the thermometer registers an excessively high temperature. The alert is audible, in the form of a signal, or an instruction, and is delivered via speaker 117. Sensor logic can further operate transceiver 119 to transmit processed sensor signals, such as vital statistics, to a remote server. The vital statistics can be securely encrypted before transmitting.

Transceiver 119 is further capable of communicating with a wireless communication device, such as a cellular telephone/PDA. For instance, transceiver 117 can be a ZIGBEE™ or BLUETOOTH® transceiver. The wireless communication device can therefore send and receive audio signals from microphone 115 and speaker 117. This “headset” operation is managed by communication logic 116, for instance, by pairing with known wireless communication devices. Communication logic 116 may further be employed to send signals from the sensors to the wireless communication device.

Alternatively, incoming audio is processed by hearing aid logic 118. This logic operates like currently used hearing aids, such as behind the ear (BTE) or in the ear (ITE) aids. Hearing aid logic 118 is programmed to adjust all processing characteristics based on an individual user's hearing profile. This programming automatically and adaptively adjusts an incoming audio signal by eliminating acoustic feedback (whistling), reducing background noise, detecting and automatically accommodating different listening environments, etc. Hearing aid logic may further control additional microphones such as microphone 115 to improve spatial hearing, transpose frequencies, and implement many other features. Furthermore, control signals in a hearing aid worn on one ear can be sent wirelessly to hearing aid logic 118 worn on the opposite ear to ensure that the audio in both ears is matched. Hearing aid logic 118 can additionally be paired with communication logic 116 to enable voice activated commands on wireless communication devices as well as processing incoming audio from external devices. Hearing aid logic 118 can also be programmed to signal alerts to the user generated by sensor logic 114.

As mentioned herein, the sensors are small, discreet, and non-invasive. Glucose sensor 121 measures a level of blood glucose in the body using one of several non-invasive methods known in the art, for instance by using a low electric current to pull glucose through the skin, or by shining an IR or near-IR beam on the skin. IR or near-IR spectroscopy uses high performance photo-resistors, photo-transistors, photo-diodes, or any combination of these. Based on a change in refractive index and hence the scattering coefficient of the blood glucose, one can estimate the change in glucose concentration. This variation can be determined by passing a modulated laser beam and measuring the phase. Variations may be processed as electric signals by sensor logic 114. Similarly, pulse oximeter 125 measures variations in arterial blood by sensing the rate of absorption of one or more beams of IR light of different wavelengths shone through the skin. Temperature sensor 123 is any sensor that registers a signal upon thermal contact with a surface, such as a temperature transducer, thermistor, thermocouple, semiconductor, etc. Sensor 123 may be compact enough to incorporate within an earpiece including speaker 117, therefore sensing the temperature from within the ear canal.

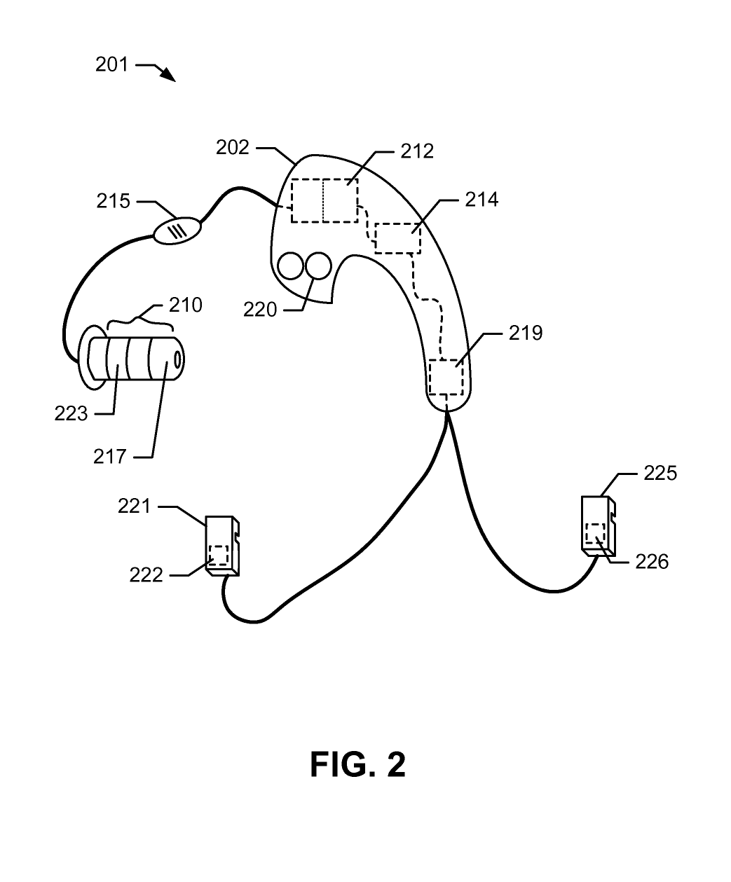

FIG. 2 shows a health monitoring device 201, according to an exemplary embodiment of the present invention. Behind-the-ear portion 202 houses microcontroller 212, logic 214, and a transceiver 219, including an antenna. Operation of device 201 is user-controlled by buttons 220. Behind the ear portion 202 is electrically coupled to the components of in-ear portion 210, which include speaker 217 and temperature sensor 223. Behind the ear portion 202 is further coupled to sensors 221 and 225, which are pulse oximeters and glucose meters, respectively. Sensors 221 and 225 may operate in a similar manner, i.e. via IR or near-IR light, or may operate differently. Sensors 221 and 225 can further have their operation controlled by logic 222 and 226, respectively. In one embodiment, the functionality of both types of sensors is included in sensor 221, and sensor 225 is optional, or non-existent.

Portion 202 is designed to fit snugly behind a user's ear. In-ear portion 210 is similarly designed to snugly fit within a user's ear canal. Sensors 221 and 225 may clip onto a user's ear lobe or any part of the ear where circulation is sufficient to generate a pulse and blood oxygen reading. Glucose meter 225 may alternatively be discreetly placed near any suitable part of the ear or neck. The wiring between the portions is designed to be as unobtrusive as possible, and can include the feature of being automatically retractable. Alternatively, in-ear portion 210 may be connected to portion 202 via a hinge, with sensors 221 and 225 being similarly physically coupled to portions 202 or 210 without using wires. Other configurations are possible. Further, the external portions of device 201 can be ornamentally designed so as to resemble jewelry or accessories, in order to remain as unobtrusive as possible.

A microphone 215 senses acoustic vibrations and transmits a corresponding audio signal to microcontroller 212. The microphone may be an acoustic transducer incorporated within a connecting wire. Alternatively, the microphone may be embedded within in-ear portion 210, for instance, a contact microphone that senses physical vibrations from a contact point within the ear canal, or even mounted upon behind-the-ear portion 202. Multiple microphones are therefore within the scope of this invention, signals from which are transmitted to, inter alia, a hearing aid logic within microcontroller 212. The hearing aid logic adjusts the audio signal and amplifies portions of the signal per a profile unique to the user, as described above.

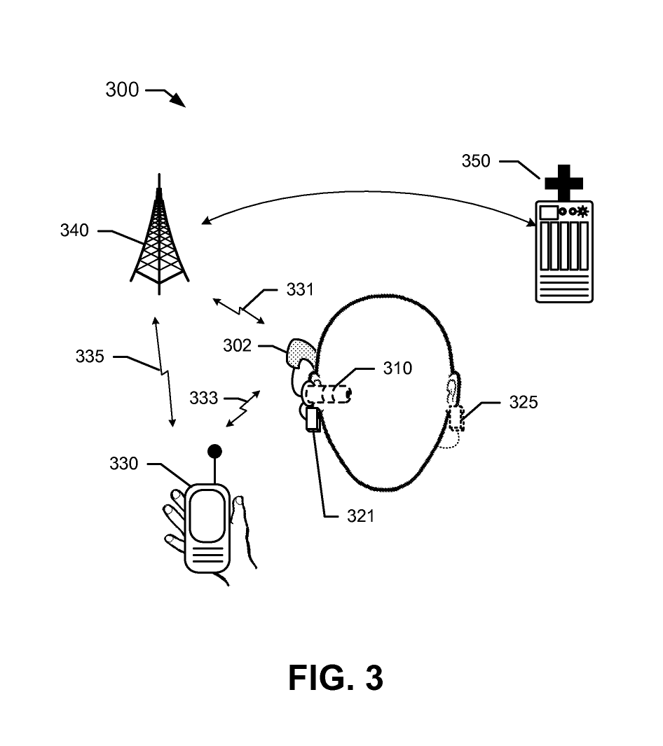

FIG. 3 shows a system 300 for monitoring a user's health, according to an exemplary embodiment of the present invention. System 300 includes a device comprised of behind-the-ear portion 302, in-ear portion 310, and sensors 321 and 325. System 300 further includes a wireless communication device 330, a base station 340, and a remote server 350. Portions 302 and 310 are respectively designed to be inserted/fit in a user's ear, and sensors 321 and 325 clip on to the user's ear lobes or any other part where a vital statistic can be measured. Portion 302 includes a transducer capable of communication with base station 340 via channel 331. Channel 331 may be a Wi-Fi or similar radio channel, or a cellular channel. In the case of a Wi-Fi channel, base station 340 is a wireless access point. The device also communicates with wireless communication device 330, via channel 333. Channel 333 uses a direct radio interface such as ZIGBEE™ or BLUETOOTH® or equivalent. There may be a provision for a wired connection as well. Wireless communication device 330 includes a transceiver to communicate with base station 340 across channel 335. Channel 335 can be any radio GSM, CDMA, UMTS, LTE, Wi-Max, or equivalent wireless communication standard used by cellular telephones. Base station 340 also functions as a gateway to a wide area network such as the internet. Base station 340 is in communication across the network with a remote server 350. Remove server 350 is part of a remote monitoring center in a medical facility, or is at least equipped with medical logic capable of generating an alert.

In-ear portion 310 includes at least a speaker, and one or more sensors such as a thermometer. A microphone may be included within in-ear portion 310, within portion 302, or included in one of the connecting wires. Multiple microphones may be included. The sensors measure vital statistics, such as temperature, and transmit the generated signals to a processor housed in portion 302. Sensors 321 and 325 similarly generate signals corresponding to vital statistics such as a pulse, blood oxygen, and blood glucose. These signals are processed by logic on a memory in communication with the processor. Alternatively, the signals may be communicated to wireless communication device 330, or to base station 340, and eventually to remote server 350. The signals may additionally be downloaded to wireless communication device 330.

An alerting logic unit analyzes the signals to determine whether or not a vital statistic exceeds a threshold. The threshold may be defined by a medical professional in consultation with the user, or by a service provider, etc. Thresholds may be unique to the user, or general in nature. If a threshold is exceeded, whether it is a maximum threshold (such as a temperature exceeding 104° F., the point of delirium), or a minimum (to prevent, for instance, hypothermia). Whereupon, an alert is generated. The alert is transmitted to the user via a speaker in in-ear portion 310. Alternatively, the alert may be transmitted to a medical professional, or an emergency service provider. In some embodiments, the alert may be sent via base station 340 to the user's wireless communication device 330, which indicates on its screen or via BLUETOOTH connection 333 that an alert has been generated. The alert may simply be a notice that the specific vital statistic threshold has been exceeded. The alert can further include a suggestion for the user to perform a specific action to send the vital statistics back to normal. For instance, an irregular pulse could generate an alert for the user to breathe slowly and deeply. An abnormal blood sugar level could generate an alert for the user to take an insulin shot. Alerts include submitting alerts to other parties or individuals known to the user, or to medical or emergency professionals. The alerting logic can be programmed in the remote server 350, the mobile communication device 330, the in-ear device, or can be distributed among these and other network elements.

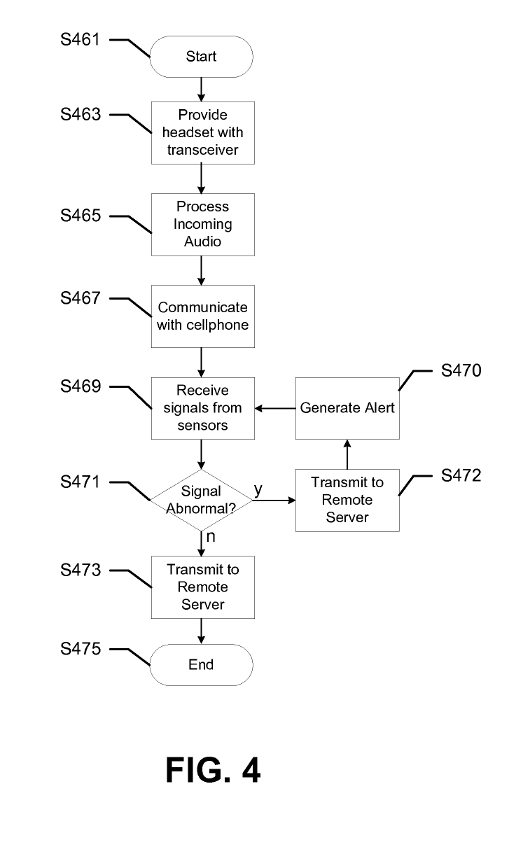

FIG. 4 shows a method for monitoring health, according to an exemplary embodiment of the present invention. The method begins S461 and a headset is equipped with a transceiver S463. The headset may be a behind-the-ear or in-ear style, and includes at least a speaker, a microphone, and a microcontroller. The headset has a logic that processes incoming audio S465 in order to make the audio audible for a user. The logic may be a hearing aid logic. The headset is capable of communicating with a wireless mobile device S467, such as a cellular telephone. The headset is further capable of communication with a plurality of sensors S469, some of which are incorporated into the headset itself, and others communicate via wires. Sensors such as thermometers, pulse oximeters, and glucose meters respectively measure temperature, pulse, blood oxygen, and blood sugar of a user. These measurements are made by sensors discreetly placed in or around the ear, and are taken in a non-invasive manner, i.e. no piercing of the skin is involved.

Measurements received from the sensors are processed and compared with desired or threshold values for the vital statistics being measured S471. If a signal does not exceed a threshold, the reading is transmitted to a remote server S473, or simply stored in a local database and the method ends S475. If, however, a vital statistic exceeds a maximum or minimum threshold, the value is transmitted to the remote server S472, and an alert is generated S470. The alert may be directed towards the user, or towards a medical professional or emergency medical service in communication with the remote server. Alternatively, the alert may be a helpful suggestion to the user. Meanwhile, the method continues monitoring signals received by sensors S469.

The present invention therefore provides systems, devices, and methods for remote health monitoring of vital statistics, including for diabetics, patients suffering from pulmonary diseases, sleep apnea, etc. without the need for excessively large and invasive monitoring methods. Further, the incorporation of headset functionality, communication with a wireless device, and audio processing logic for the hearing impaired add several unique features to the described embodiments. These devices, systems, and methods are usable in home health monitoring, out-patient settings, and in-patient settings including hospitals.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A wireless in-ear health monitoring device, comprising:
a processor;
a memory in communication with the processor;
a transceiver in communication with the processor, the transceiver being configured to communicate over a wireless network;
a plurality of health sensors coupled to the device, the plurality of health sensors measuring a corresponding plurality of vital statistics;
a sensing logic on the memory to operate the plurality of health sensors and to receive the corresponding plurality of vital statistics from the plurality of health sensors;
a communication logic on the memory to operate a microphone and a speaker coupled to the device; and
a hearing aid logic on the memory to process an incoming audio signal.

2. The device of claim 1, wherein the plurality of health sensors includes a thermometer, a pulse oximeter, and a blood glucose meter.

3. The device of claim 2, wherein the blood glucose meter and the pulse oximeter further comprise a Near Infra-Red (NIR) detector adapted to be clipped to an earlobe.

4. The device of claim 2, wherein the thermometer and the speaker are adapted to be inserted into an ear canal.

5. The device of claim 1, further comprising alerting logic to generate an alert when one of the plurality of vital statistics exceeds a maximum or minimum threshold.

6. The device of claim 5, wherein the alert is transmitted to a user of the device via the speaker.

7. The device of claim 5, wherein the alert is transmitted to a remote server on a network via the transceiver.

8. The device of claim 1, wherein the communications logic operates the transceiver to communicate with a wireless communication device.

9. The device of claim 8, wherein the transceiver is one of a BLUETOOTH and a ZIGBEE transceiver.

10. The device of claim 1, wherein the hearing aid logic processes an incoming audio signal based upon a hearing profile for a user of the device.

11. The device of claim 10, further comprising a second microphone to generate the audio signal that is processed by the hearing aid logic.

12. A system for monitoring health of a user, the system comprising:
a microcontroller including a processor, a memory, and a transceiver;
a plurality of health sensors in communication with the microcontroller, the plurality of health sensors measuring a corresponding plurality of vital statistics for the user;
a headset in communication with the microcontroller, the headset including a speaker and a microphone;
a hearing aid logic to process an incoming audio signal based upon a hearing profile for the user; and
an alerting logic in communication with the microcontroller to generate an alert via the speaker when one of the plurality of vital statistics exceeds a maximum or minimum threshold;
wherein the transceiver is configured to allow communication with a wireless telecommunication device.

13. The system of claim 12, wherein the plurality of health sensors includes a thermometer, a pulse oximeter, and a blood glucose meter.

14. The system of claim 13, wherein the blood glucose meter and the pulse oximeter further comprise a Near Infra-Red (NIR) detector adapted to be clipped to an earlobe.

15. The system of claim 12, wherein the thermometer and the speaker are adapted to be inserted into an ear canal.

16. The system of claim 12, further comprising a remote server on a network, wherein the transceiver communicates the plurality of vital statistics to the remote server to be analyzed by a healthcare professional.

17. The system of claim 16, wherein the transceiver communicates the plurality of vital statistics to the remote server via the wireless communications device.

18. The system of claim 12, further comprising a user interface on the wireless telecommunication device enabling a user to configure the operation of the system.

19. A method for health monitoring, comprising:
providing a headset to be worn by a user, the headset including a speaker and a microphone;
enabling communication between the headset and a wireless communication device operated by the user;
incorporating a plurality of health sensors into the headset, the plurality of health sensors measuring a corresponding plurality of vital statistics for the user;
alerting the user via the speaker if any of the plurality of vital statistics exceeds a maximum or a minimum threshold;
processing an incoming audio signal based on a listening profile for the user; and
outputting the processed audio signal via the speaker.

20. The method of claim 19, further comprising transmitting the plurality of vital statistics to a remote server for further processing.
